SEQUENCE LISTING (1 sequence)

SEQ ID NO: 1 - DNA, 8 bp, Artificial sequence
  Octamer motif consensus sequence (common regulatory domain of immunoglobulin genes)
atgcaaat

(19) United States
(12) Patent Application Publication
(10) Pub. No.: US 2004/0037821 A1
(43) Pub. Date: Feb. 26, 2004

(54) Gene-regulating conjugates

(76) Inventors: Andrea Crisanti, London (GB); Alison Mary Mortlock, London (GB)

Correspondence Address: SALIWANCHIK LLOYD & SALIWANCHIK, A PROFESSIONAL ASSOCIATION, 2421 N.W. 41ST STREET, SUITE A-1, GAINESVILLE, FL, 326066669

(21) Appl. No.: 10/311,798

(22) PCT Filed: Jun. 20, 2001
(86) PCT No.: PCT/GB01/02707

(30) Foreign Application Priority Data
Jun. 20, 2000 (GB) 0015090.4

(51) Int. Cl. A61K 38/47; C12N 9/22

(52) U.S. Cl. 424/94.61; 435/199

(57) ABSTRACT

A conjugate for controlling the expression of a gene comprises a nucleic acid-binding domain, a gene-regulating region and a factor that permits translocation of the conjugate across a cell membrane, wherein the nucleic acid-binding domain is heterologous to that naturally associated with the gene-regulating region, and binds to a conserved sequence on the gene.

### FIELD OF THE INVENTION

This invention relates to protein conjugates that may be administered to control the expression of selected genes.

### BACKGROUND OF THE INVENTION

Gene expression in multi-cellular organisms is controlled in a temporally and spatially regulated manner by a complex network of interactions involving DNA sequences and a large repertoire of nuclear proteins known as transcription factors. These proteins have the ability to bind selectively to specific DNA target sequences placed upstream of the expressed gene sequences. The binding of a transcription factor to a DNA sequence can either activate or suppress the expression of different sets of genes.

Gene expression in distinct cell types will depend on the nature of the transcription factor synthesised in that cell type and on the presence of the corresponding DNA target sequences upstream of the genes. With the notable exception of hormones that can activate or suppress individual transcription factors it has proven very difficult to manipulate the endogenous gene expression of specific cell types without introducing exogenous DNA. Currently, medically relevant endogenous gene products, such as cytokines and cell growth factors, are administered as purified recombinant proteins. This strategy has several limitations as it can only be applied to secretory proteins. The endogenous gene expression of different cell types can be modified by supplying the cells with an extra copy of the gene to be expressed under the transcriptional control of viral promoters. This approach requires the introduction of foreign DNA into the organism and the use of viral promoters to ensure a high and stable transcription rate.

WO-A-99/10376 discloses fusion proteins having a transcription activator region and a protein transduction domain for entry of the fusion protein into the cell. The fusion proteins are intended primarily as an in vitro tool to study the effect of a compound of interest on cell function. The transcription activator region is engineered to comprise a DNA-binding region and a region that activates transcription. Examples of DNA-binding proteins that are considered to be suitable are E2F-1, C-Myb, Fos, Gal4, EST1, Elf-I and T7 RNA polymerase. However, these DNA-binding proteins are specific to DNA regions which are found widely on many different genes, and therefore many different genes may be targeted by the fusion proteins.

WO-A-99/11809 relates to conjugates that contain the homeodomain of Antennapedia. The homeodomain is prepared in a way that permits translocation of proteins greater than 100 amino acids. Conjugates comprising the homeodomain and DNA-binding proteins are disclosed. However, the specific DNA-binding proteins referred to are similar to those in WO-A-99/10376 and are expected to bind to DNA regions found in many genes.

### SUMMARY OF THE INVENTION

The present invention discloses a strategy that has the potential to overcome many of the limitations in manipulating endogenous gene expression, and allow the selective induction of any given endogenous gene.

According to the invention, a conjugate for controlling the expression of a gene, comprises:

a nucleic acid-binding domain;

a gene-regulating region; and

a factor that permits translocation of the conjugate across a cell membrane;

wherein the nucleic acid-binding domain is heterologous to that naturally associated with the gene-regulating region and binds to a sequence that is specific to the target gene.

The nucleic acid-binding domain is chosen for its ability to bind selectively to a gene of interest. This localises the gene-regulating activity at an appropriate site to exert the desired effect. Typically the nucleic acid-binding domain will be capable of binding to a region upstream of the target gene. However, the nucleic acid-binding domain may be designed to bind to any suitable region, not merely known regulatory domains.

In contrast to conjugates of the prior art, the target site for the nucleic acid-binding domain is not a binding site for endogenous DNA-binding proteins, but is rather a unique site that is specific only for the target gene. The binding domain can then be designed to bind to the target site to provide selective targeting to the gene.

According to a second aspect of the invention, a conjugate of the invention is used in therapy, in particular, the manufacture of a medicament for endogenous regulation of gene expression.

According to a third aspect of the invention, a conjugate is prepared by first selecting a conserved or unique DNA sequence upstream of a target gene, screening an array of potential DNA-binding peptides (or proteins) for affinity to the selected sequence and selecting a peptide with affinity, and producing the conjugate with the selected peptide as the nucleic acid-binding domain.

The present invention enables the conjugates to target selectively specific genes. It is possible to select a conjugate to target a particular gene (e.g. via an enhancer or promoter sequence), to deliver a gene-regulator that would not otherwise be associated with the target gene. Many enhancer or promoter sequences on a gene are cell-specific, for example the immunoglobulin enhancer functions only in B-lymphocytes. Using conjugates of the invention, however, it may be possible to deliver a wide range of different gene regulators to the immunoglobulin genes.

### DESCRIPTION OF THE DRAWINGS

The invention is described with reference to the accompanying drawings where:

FIG. 1 illustrates an expression plasmid that codes for a fusion protein of Antennapedia and the tetracycline repressor; and

FIG. 2 illustrates a reporter plasmid that comprises the luciferase gene under the control of a tetracycline promoter sequence.

### DESCRIPTION OF THE INVENTION

The conjugates of the present invention are designed with three distinct regions: a first region comprising a factor that permits translocation of the conjugate across a cell membrane; a second region comprising a gene-regulating domain; and a third region comprising a nucleic acid-binding domain.

The first region may comprise any factor that permits translocation across a cell membrane. Many factors having this capability are known, in particular the homeodomain of antennapedia, the VP22 from herpes simplex virus, and tat from HIV have all been well characterised as translocating factors. Preferably, there should be a sequence that functions as a nuclear localisation signal to target the conjugate to the nucleus. Nuclear localisation signals are well known to the skilled person.

In a preferred embodiment of the invention, the translocation factor is derived from the homeodomain of antennapedia.

The homeodomain of the Antp gene obtainable from Drosophila is disclosed in WO-A-99/71809. Sequences homologous to this homeodomain have been isolated from other organisms, including vertebrates, mammals and humans, and these are included in the present invention. The homeodomain may be prepared using standard techniques such as cloning using the procedure described in Joliet et al., PNAS, 1991; 88:1864-1868. In addition, WO-A-99/11809 discloses the preparation of Antp constructs that are capable of translocating proteins larger than 100 amino acids in length. This is a preferred method of preparing the Antp constructs of the present invention.

Although the Antp sequence in multicellular organisms is generally conserved in nature, this may not necessarily be the case and other such sequences are included in the present invention. For example, the translocating factor may have sequence identity from about 50% or more, e.g. 60%, 70%, 80% or 90%, with the sequence obtainable from Drosophila. Sequence identity may be determined using such commercially available programmes as GAP.

In addition, synthetic variants may be used provided that they retain the ability to translocate across the membrane. Synthetic variants will generally differ from the naturally-occurring proteins by substitution, particularly conservative substitution. By conservative amino acid changes we mean replacing an amino acid from one of the amino acid groups, namely hydrophobic, polar, acidic or basic, with an amino acid from within the same group. An example of such a change is the replacement of valine by methionine and vice versa.

In a separate preferred embodiment, the translocating factor is histone, or is a functional fragment of histone. Histone fragments that act as translocation factors are disclosed in Zaitsev et al., Gene Therapy, 1997; 4: 586-592 and in co-pending International Patent Application No. PCT/GB01/01699.

The second region of the conjugate comprises a gene-regulating factor. The gene-regulating factor may be either an activator of gene expression, or a repressor of expression. Preferably, the factor is an enhancer of expression. The factor may be a protein or may be a polynucleotide, e.g. a promoter or enhancer sequence that can be used to enhance expression of a gene. The factor may therefore be a promoter nucleic acid sequence that functions more effectively than that associated endogenously with the target gene. Suitable activators are disclosed in WO-A-99/10376. Many gene-regulating factors are known. For example, nuclear protein Oct-1 is well characterised as an activator of gene transcription. This factor is specific for an octamer motif having the consensus sequence ATGCAAAT, which is a common regulatory domain of immunoglobulin (Ig) genes.

An alternative activator is the herpes simplex virus vision protein 16 (VP16), the amino acid sequence of which is disclosed in Triezenberg et al., Genes Dev., 1988; 2: 718-729.

The gene-regulating factor must exert its effect from the position on the gen sequence at which the conjugate binds. It will be appreciated that suitable t sts can be carried out to monitor the levels of expression, and the results compared with a control system.

The gene-regulating factor may exert its effect directly on the genomic DNA, or may act to control gene function indirectly, for example by targeting a component necessary for expression.

The target site (target gene) may be any gene sequence, the expression of which needs to be regulated. The gene sequence may be, for example, a mutant gene (oncogene), the expression of which is to be repressed. In addition, the gene sequence may be viral DNA that is incorporated into a host genome. It is desirable to repress certain integrated viral genes, as these may be implicated in pathogenesis. The overexpression of certain genes can also result in disease, and so selective targeting and repression of the genes are desirable. For example, the over-expression of growth factors or hormones is generally undesirable, and so control of gene expression is a useful therapy.

The under-expression of genes can also result in disease, due to a lack of endogenous product. It is therefore desirable to enhance expression of these genes, to correct the defect. For example, the gene of interest may encode a product used in metabolism, and so the correct expression of the gene is necessary to maintain a healthy metabolic function.

The third region comprises a nucleic acid-binding domain. The nucleic acid-binding domain may be a DNA-binding domain or an RNA-binding domain. Typically, the domain will be DNA-binding, however, in the context of RNA-binding, the domain will target, typically, nascent RNA being transcribed from DNA at the selected site. The nucleic acid-binding domain is chosen on the basis of its ability to bind to a selected sequence on, or associated with, the gene of interest. The selected sequence is not an endogenous binding site for transcription factors, but is selected for its specificity to that gene of interest. The targeted sequence is not necessarily a regulatory region and the binding domain may be engineered to bind to alterative sequences that are highly conserved in the gene of interest. The binding domain will be heterologous to that naturally associated with the gene-regulating factor.

The use of the term “conserved” in the context of the target gene sequence is intended to refer to a target sequence that is specific for that gene, i.e. not found in other unrelated genes. The sequence will usually be greater than 6 nucleotides, preferably greater than 8 nucleotides, more preferably greater than 10 nucl otides, and most preferably 16 or more nucleotides. Suitable target sequences can be identified using conventional sequence analysis software programmes, with comparisons to other gene sequences being accomplished based on the sequence information made available as part of the Human Genome Project. For example, having chosen the gene to be targeted, the gene sequence can be analysed using conventional computer programmes, to identify a sequence that is specific for that gene. This sequence is then used as the target to design suitable binding proteins.

Examples of molecules that bind to DNA include proteins with a zinc finger motif or a leucine zipper motif or proteins with a helix-turn-helix motif. The binding domain may also be derived from suitable regulatory proteins, i.e. either positive regulators or negative regulators. For example, the binding domain may comprise the appropriate DNA-binding domain from a λ repressor protein, e.g. λ Cro. Alternatively the suitable regions of protamine may be used. These known DNA-binding proteins are modified or engineered to have unique binding affinity for the target sequence.

The binding domain may be selected by using conventional techniques. Once the conserved gene sequence has been selected, this can be used as the target in an assay to select suitable binding proteins or peptides. Conventional binding proteins may be adapted/modified using recombinant DNA techniques, to produce proteins that bind specifically to the conserved sequence.

A particularly suitable technique is phage display, a review of which is given in Cannon et al., IVD Technology, 1996; November/December: 22-31. Phage display is an efficient way of producing large numbers of diverse proteins/peptides, and selecting those that bind to a particular target. Alternative techniques, for example, ribosome display, may also be used to select those proteins that bind to the conserved sequence.

In addition, Moore et al., Proc. Natl. Acad. Sci., 2001; 98(4): 1432-1436, and Moore et al., Proc. Natl. Acad. Sci., 2001; 98(4): 1437-1441, show that polyzinc finger peptides can be adapted to produce “designer peptides” that have novel binding specificities. These publications show that it is possible to design peptides that bind to unique sites on a genome. In a preferred embodiment, the nucleic acid-binding domain is a multi-zinc finger peptide that binds to a unique DNA sequence on or at the target gene sequence. Preferably there are at least four, more preferably six zinc fingers that make up the binding domain.

Methods for producing the conjugate according to the present invention, will be apparent to those skilled in the art. In particular, the production of fusion proteins is well known and methods for constructing suitable gene constructs that express the desired conjugate in a suitable host system, are well known.

The three regions will all be functional when they are part of the conjugate. In addition, the regions may be presented on the conjugate in any order.

In the preferred embodiment, the conjugate is a fusion protein and is produced by ligating the DNA molecules that encode each component of the construct, to produce a hybrid DNA molecule. When expressed in a host cell, e.g. in a bacterial cell or a baculovirus system, the hybrid DNA molecule is expressed and the fusion protein is produced. The hybrid DNA molecule may also contain promoter or enhancer sequences that aid expression.

Conjugates of the present invention may be used in the manufacture of a pharmaceutical composition to treat a disease. The composition may optionally comprise a pharmaceutically acceptable carrier, diluent, excipient or adjuvant. The choice of pharmaceutical carrier, excipient or diluent can be selected with regard to the intended route of administration and standard pharmaceutical practice.

The pharmaceutical compositions can be administered by any suitable route. In particular, oral, transdermal, parenteral or mucosal delivery may be appropriate.

The conjugates may be used to treat any animal, in particular humans. Veterinary applications are also intended. The appropriate dosage can be selected according to various factors that are known to those skilled in the art.

When administered, the conjugate will localise at the desired gene sequence to effect gene regulation. In a preferred embodiment, the conjugate targets genes which express products having a beneficial effect on the organism. In one example, the target gene expresses erythropoietin, the expression of which may be regulated, thereby promoting the production of additional erythropoietin in the patient.

Although intended primarily for therapeutic or prophylactic use, the conjugates of the invention may be used in diagnostic applications, e.g. in vitro assays intended to study the expression of a particular gene, or to investigate the function of a gene.

The following Example illustrates the invention.

In the following Example, the homeodomain of the Antennapedia protein (Antp) was fused to the C terminus of the tetracycline repressor (TetR) from E. coli. Incubating the fusion protein with HeLa cells resulted in delivery of the fusion protein to the nucleus. To assess gene regulation following delivery, HeLa cells were also incubated with a luciferase reporter plasmid containing a TetR-regulatable promoter. Luciferase expression was repressed in cells incubated with the fusion protein.

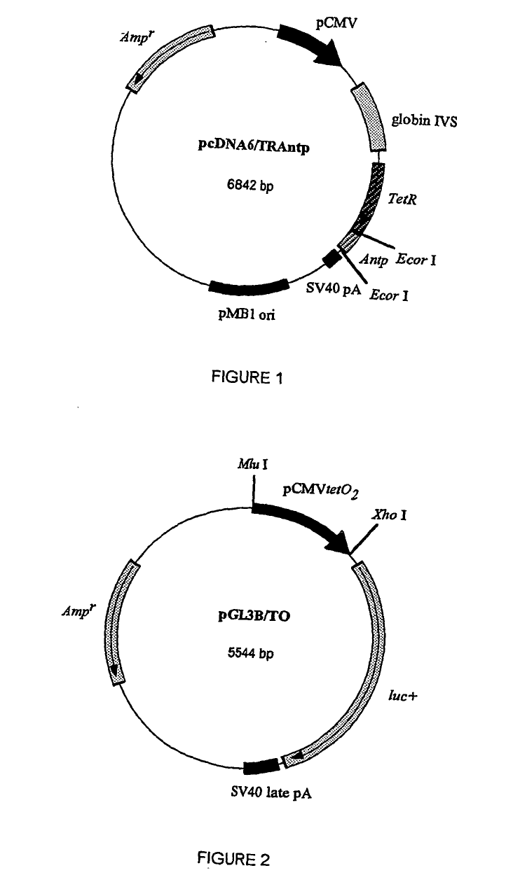

In order to express a TetRAntp fusion protein in HeLa cells, the 180 bp Antp homeobox domain was cloned into the plasmid pcDNA6/TR (Invitrogen) to create pcDNA6/TRAntp (FIG. 1). Antp was fused to the C-terminus of TetR, since the DNA binding region of TetR is located at the N-terminus of the protein. Antp was amplified by PCR from a template plasmid using conventional techniques and cloned in-frame into an EcorI site at the C-terminus of TetR. The resulting TetRAntp fusion was confirmed by sequencing.

To assess the level of expression by TetR or TetRAntp fusion protein, a reporter plasmid was constructed that expresses luciferase under the control of the pCMVtetO₂ promoter. The pGL3-Basic reporter plasmid (Promega) contains a modified cytosolic form of the firefly (Photinus pyralis) luciferase gene (luc+). In order for luciferase to be expressed, pGL3-Basic requires the insertion of a functional eukaryotic promoter in the correct orientation. The CMV promoter (pCMVtetO₂) containing two tetracycline (Tc) operator (tetO₂) sequences inserted between the TATA box and transcription start site was obtained from pcDNA4/TO (Invitrogen). A 726 bp fragment containing pCMVtetO₂ was excised MluI to XhoI and cloned into pGL3Basic to create pGL3B/TO (FIG. 2).

Insertion of pCMVtetO₂ resulted in a high level of luciferase expression from the functional promoter.

In cells transfected with a 6:1 ratio of pcDNA6/TRAntp:pGL3B/TO, luciferase expression was repressed in the absence of Tc. When Tc was added to the culture medium, luciferase expression was induced 6-fold. Repression by TetRAntp was very similar to repression by TetR alone. Induction with Tc resulted in a 6-fold increase in luciferase expression.

Assuming TetR and TetRAntp are expressed at similar levels, these results suggest that Antp fused to the C-terminus of TetR has no effect upon the responsiveness of TetR to Tc, or on the ability of TetR to repress transcription. The fusion protein was therefore able to translocate and to select the appropriate site for transcription repression. The results show that the targeted repression of a selected gene is possible, and it is expected that by modifying the DNA-binding component, a versatile array of repressor molecules can be produced, to target selectively different endogenous genes.

1. A conjugate for controlling the expression of a gene, comprising:
a nucleic acid-binding domain;
a gene-regulating region; and
a factor that permits translocation of the conjugate across a cell membrane;
wherein the nucleic acid-binding domain is heterologous to that naturally associated with the gene-regulating region and binds to a sequence that is specific to the gene.

2. A conjugate according to claim 1, wherein the conserved sequence is not found in a heterologous gene.

3. A conjugate according to claim 1 or claim 2, wherein the nucleic acid-binding domain is capable of binding to an upstream regulatory domain of the gene.

4. A conjugate according to any preceding claim, wherein the nucleic acid-binding domain comprises a zinc-finger motif.

5. A conjugate according to any preceding claim, wherein the gene-regulating region is an enhancer of gene expression.

6. A conjugate according to any preceding claim, wherein the nucleic acid-binding domain is a DNA-binding domain.

7. A conjugate according to any preceding claim, wherein the translocation factor comprises the homeodomain of antennapedia, or a functional variant thereof.

8. A conjugate according to any preceding claim, for use in therapy.

9. A polynucleotide that encodes a conjugate according to any preceding claim.

10. Use of a conjugate according to any preceding claim, in the manufacture of a medicament for a condition which can be affected by endogenous regulation of gene expression.

11. Use according to claim 10, wherein gene expression is enhanced.

12. Use according to any preceding claim, wherein the gene expresses erythropoietin.

13. A method for the production of a conjugate according to any of claims 1 to 7, comprising selecting a DNA sequence that is specific to a target gene, screening an array of potential DNA-binding peptides or proteins for affinity to the conserved sequence, selecting a peptide or protein with affinity and producing the conjugate with the selected peptide or protein as the DNA-binding domain.
